(19) United States
(12) Patent Application Publication
(10) Pub. No.: US 2009/0233935 A1
(43) Pub. Date: Sep. 17, 2009

(54) Methods for Fabrication of Antimicrobial Surfaces

(75) Inventor: Robert Engel, Carle Place, NY (US)

Correspondence Address: HOFFMANN & BARON, LLP, 6900 JERICHO TURNPIKE, SYOSSET, NY, 11791, US

(73) Assignee: Research Foundation of City University of New York, New York, NY (US)

(21) Appl. No.: 12/083,478

(22) PCT Filed: Oct. 12, 2006
(86) PCT No.: PCT/US2006/040587

§ 371 Date: Apr. 11, 2008

Related U.S. Application Data
(60) Provisional application No. 60/726,596, filed on Oct. 14, 2005.

(51) Int. Cl. A61K 31/504; A61K 31/497; A61K 31/13

(52) U.S. Cl. 514/249; 514/252.12; 514/579

(57) ABSTRACT

A method for rendering a carboxyl-containing polymer surface resistant to microbial growth, the method comprising: (i) reacting the carboxyl-containing polymer surface with a metal borohydride in a solvent system comprising a water-soluble alcohol having one to three hydroxy groups and one to four carbon atoms, and optionally up to about fifty percent water, in a temperature range having a minimum of about 60° C. and a maximum of about 80° C., and for an amount of time sufficient for reducing carboxyl groups on the carboxyl-containing polymer surface to surface hydroxymethyl groups without altering the nature of the bulk of the polymer; and either: (iia) converting hydroxy groups of the surface hydroxymethyl groups to leaving groups and displacing the leaving groups with one or a combination of antimicrobial agents; or (iib) displacing the hydrogen atoms of the hydroxy groups of the surface hydroxymethyl groups with one or a combination of antimicrobial agents.

### CROSS-REFERENCE TO RELATED APPLICATION

This application claims the benefit of U.S. Provisional Application No. 60/726,596, filed Oct. 14, 2005, which is incorporated herein by reference.

### BACKGROUND OF THE INVENTION

Current fears of antibiotic-resistant bacteria and other microbes as well as of bioterrorism have increased the importance of developing new ways to protect people from microbial infection. It is, for example, important to develop new materials for making clothing that can be more safely worn in contaminated environments. Such materials would be useful, for example, in hospitals and during military and civilian operations where bacterial contamination has occurred, or is expected.

U.S. Patent Application Publication 2005/0181006 A1 to Engel, et al. is directed to antimicrobial surfaces. The antimicrobial surfaces of Engel et al. are prepared by treating a solid surface naturally having hydroxyl groups in the unmodified state (e.g., carbohydrates) with a charged antimicrobial agent.

However, it would be highly beneficial to render antimicrobial other surfaces which do not naturally contain hydroxyl groups. Such surfaces typically lack functional groups (e.g., hydroxyl groups) by which antimicrobial agents can attach. The introduction of hydroxyl groups into such materials (e.g., polyester fabrics) to make them suitable as substrates for the attachment of antimicrobial agents is not straight-forward. For example, polyester fabrics will degrade under most conditions which are typically used for the reduction of polyester groups into hydroxyl groups.

Accordingly, there is a need for new methods for rendering such non-hydroxyl-containing surfaces antimicrobial. Such surfaces often comprise carboxyl groups. There is, therefore, a particular need for rendering carboxyl-containing materials, such as polyester fabrics, antimicrobial.

### SUMMARY OF THE INVENTION

These and other objectives as will be apparent to those having ordinary skill in the art have been achieved by providing a method for rendering a carboxyl-containing polymer surface resistant to microbial growth, the method comprising:

(i) reacting the carboxyl-containing polymer surface with a metal borohydride in a solvent system comprising a water-soluble alcohol having one to three hydroxy groups and one to four carbon atoms and optionally up to about fifty percent water, in a temperature range having a minimum of about 60° C. and a maximum of about 80° C., and for an amount of time sufficient for reducing carboxyl groups on the carboxyl-containing polymer surface to surface hydroxymethyl groups without altering the nature of the bulk of the polymer; and either

(iia) converting hydroxy groups of the surface hydroxymethyl groups to leaving groups and displacing the leaving groups with one or a combination of antimicrobial agents; or

(iib) displacing the hydrogen atoms of the hydroxy groups of the surface hydroxymethyl groups with one or a combination of antimicrobial agents;

the antimicrobial agents being positively charged after displacing the leaving groups or the hydrogen atoms of the hydroxy groups.

### DETAILED DESCRIPTION OF THE INVENTION

The invention relates to methods for rendering carboxyl-containing polymer surfaces resistant to microbial growth. The carboxyl-containing polymer surfaces can be a part of, for example, clothing, bandages, sutures, protective gear, containers, and the like. In a preferred embodiment, the carboxyl-containing polymer is a carboxyl-containing fabric, such as a polyester.

The method comprises, first, selectively reducing carboxyl groups on the carboxyl-containing polymer surface to surface hydroxymethyl groups without altering the nature of the bulk of the carboxyl-containing polymer. The method achieves the selective conversion by reacting the carboxyl-containing polymer surface with a metal borohydride under specific reaction conditions.

The metal borohydride comprises a metal ion (Mⁿ⁺) associated with an anionic borohydride complex (BH₄⁻) wherein n can be any suitable value, and preferably, 1 or 2. More preferably, the metal borohydride is an alkali metal borohydride. Some examples of alkali metal borohydrides include lithium borohydride, sodium borohydride, and potassium borohydride. The metal borohydride may also be a combination of metal borohydrides.

The reaction conditions include a solvent system which comprises a water-soluble alcohol having one to three hydroxy groups and one to four carbon atoms, and optionally, up to about fifty percent water. Some examples of such alcohols include methyl alcohol, ethyl alcohol, isopropyl alcohol, t-butyl alcohol, ethylene glycol, glycerol, and combinations thereof. The water content can be, for example, exactly or approximately 0%, 5%, 10%, 20%, 30%, 40%, 45%, or 50%.

Preferably, the temperature range used in the method has a minimum of about 60° C. and a maximum of about 80° C. For example, the method can be practiced at a minimum temperature of about 60° C., 61° C., 63° C., 65° C., 67° C., 69° C., 70° C., or 73° C., and a maximum temperature of about 74° C., 75° C., 76° C., 77° C., 78° C., 79° C., or 80° C. Particularly preferred temperature ranges are derived by combining any of the above minimum temperatures with any of the above maximum temperatures.

The amount of time used for treating the carboxyl-containing polymer surface under the conditions described above varies according to the temperature used. For example, higher temperatures require shorter amounts of time. At a particular temperature, a minimum reaction time is required for sufficient conversion of the surface carboxyl groups to surface hydroxymethyl groups. In addition, at a particular temperature, a maximum reaction time is required in order to prevent excessive degradation of the carboxyl-containing polymer.

Typically, a minimum of approximately two hours and a maximum of about four hours for a temperature of about 60° C. are sufficient reaction times. For a temperature of about 80° C., a minimum of approximately fifteen minutes and a maximum of about thirty-five minutes are sufficient. As the temperature increases from about 60° C. to about 80° C., a sufficient amount of time is within a range having a minimum decreasing from about two hours to about fifteen minutes and a maximum decreasing from about four hours to about thirty-five minutes.

After the carboxyl groups in the polymer surface have been reduced to hydroxymethyl groups, the hydroxyl-containing surface is reacted with one or a combination of antimicrobial agents to attach the antimicrobial agents to the surface by methods known in the art. Preferably, the attachment of antimicrobial agents is achieved by either of two methods.

According to one method, the surface hydroxyl groups are converted to leaving groups and the leaving groups displaced under suitable conditions by an antimicrobial agent having one or more nucleophilic groups. Such reactions are well known in the art. See, for example, Introduction to Organic Laboratory Techniques, Pavia, Lampman, Kriz, Second Edition, Saunders College Publishing, ©1982.

For example, in one embodiment, the surface hydroxyl groups are converted to ester leaving groups. Some examples of ester leaving groups include sulfonyl and acyl groups.

The hydroxyl groups can be converted to ester groups by reacting the hydroxyl groups with a suitable activating compound. Some examples of classes of activating compounds include sulfonyl chlorides, acyl chlorides, and organic esters. Some examples of sulfonyl chlorides include benzenesulfonyl chloride, p-toluenesulfonyl chloride (i.e., tosyl chloride, TsCl) and methyl sulfonyl chloride (mesyl chloride, MsCl). Some examples of acyl chlorides include acetyl chloride and benzoyl chloride. Some examples of organic esters include ethyl benzoate, methyl acetate, and ethyl acetate.

A polyester fabric can be treated with an activating compound in a suitable solvent. The solvent must not degrade the bulk of the carboxyl-containing material.

The amount of reagent and volume of suitable medium are known to, or can easily be determined by, those skilled in the art. Some solvents which may be used for carrying out the reaction include, but are not limited to, pyridine, hexane, heptane, ether, toluene, ethyl acetate, and mixtures thereof.

Activation with a sulfonyl chloride or acyl chloride requires a proton-sink. Accordingly, activation is conveniently accomplished in an aqueous bicarbonate medium. Alternatively, an amine-based proton-sink, e.g., pyridine or a polymeric tertiary amine, can be used. An example of a polymeric tertiary amine, includes, for example, DEAE-cellulose.

Activation of a hydroxyl-containing surface, as prepared above, with an organic acid can be accomplished, for example, in the presence of a catalytic amount of an inorganic acid in a suitably polar non-reactive solvent. The solution containing the surface, e.g., a polyester fabric, can also be heated, if necessary, to a suitable temperature for promoting the reaction. The temperature should be chosen so as not to degrade the polyester fabric (preferably, not more than 80° C.). The organic acid can be, for example, benzoic acid or acetic acid. The inorganic acid can be, for example, sulfuric, hydrochloric, or nitric acid.

The non-reactive solvent must not appreciably react with the organic acid and must not appreciably hydrolyze under the conditions used. Some suitable non-reactive solvents include higher boiling ethers, such as ethylene glycol dimethyl ether (glyme), bis-(2-methoxyethyl)ether, and tetrahydrofuran.

In another embodiment, the surface hydroxyl groups are converted to halide leaving groups. The hydroxyl groups can be converted to halide groups by treating the surface with a compound capable of converting a hydroxyl group to a halide group. Some examples of compounds capable of converting surface hydroxyl groups to chloro and bromo groups include thionyl chloride and phosphorus tribromide, respectively.

In yet another embodiment, the surface hydroxyl groups are converted to hydronium ion leaving groups. The hydroxyl groups can be converted to hydronium ion groups by, for example, reacting the hydroxyl groups with an inorganic acid, such as a hydrogen halide (e.g., hydrochloric or hydrobromic acids), sulfuric acid, or nitric acid in an aqueous-based solution. Alternatively, the hydroxyl groups can be reacted with an organic acid, such as acetic, propanoic, or benzoic acid, under conditions which do not favor esterification.

The number of hydroxyl groups on a polymer surface depends on the number of carboxyl groups reduced and the size of the surface. For example, the number of hydroxyl groups on a polymer surface is more than one, typically more than a thousand, more than ten thousand, or more than one hundred thousand.

It is not necessary to convert all of the available hydroxyl groups (i.e., sites) present on the carboxyl-containing surface to leaving groups. For example, less than about 10% of the available hydroxyl groups on a surface may be converted to subsequently provide sufficient antimicrobial activity. Preferably, about 25%, more preferably about 50%, and most preferably about 75% of the available hydroxyl groups may be converted.

After conversion of the surface hydroxyl groups to leaving groups, the leaving groups are then displaced by a suitable nucleophilic group on an antimicrobial compound. Some examples of suitable nucleophilic groups capable of displacing leaving groups include amino, mercapto, and phosphino groups. Some examples of suitable amino groups include —NH₂, —N(CH₃)H, —N(CH₃)₂, —N(CH₂CH₃)₂, —N⁺(CH₃)₂(CH₂CH₂)N(CH₃)₂, piperadinyl, pyridinyl, piperazinyl, pyrazinyl, and 1,4-diazabicyclo[2.2.2]octanyl groups. Some examples of mercapto groups include —SH, —SCH₃, tetrahydrothiopyranyl, 1,4-dithianyl, thiophenyl, and thiophenyl groups. Some examples of phosphino groups include —P(CH₃)₂, —P(CH₂CH₃)₂, —P(C₆H₅)(CH₃), —P(C₆H₅)₂, and —P⁺(CH₃)₂(CH₂CH₂)P(CH₃)₂ groups.

In a preferred embodiment, the activated carboxyl-containing surfaces are rendered antimicrobial by the chemical attachment of a suitable tertiary amine, thioether, or tertiary phosphine species in a suitable reaction medium. Some examples of suitable reaction media include, aqueous ethanol, ethanol, methanol, 2-propanol, acetonitrile, propionitrile, and mixtures thereof.

Examples of particularly suitable tertiary amine, tertiary phosphine, and thioether species include:

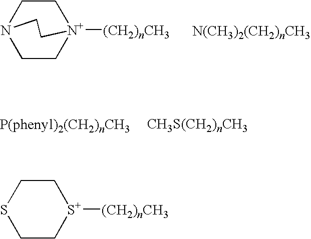

wherein n is 9 to 23.

According to another method for attaching antimicrobial compounds to surface hydroxyl groups, the surface hydroxyl groups are reacted with one or a combination of antimicrobial agents capable of displacing the hydrogen atoms of the hydroxyl groups. For example, an ester group or activated ester group on an antimicrobial agent can undergo a condensation reaction with the surface hydroxyl groups. The conditions suitable for creating the resulting ester bonds are well known in the art, as described above.

Such a condensation reaction can be accomplished by, for example, treating a hydroxyl-containing polyester fabric, as prepared above, with a carboxylic acid derivative of an antimicrobial agent in the presence of a catalytic amount of an inorganic acid in a suitably polar non-reactive solvent. The carboxylic acid group can additionally be activated, e.g., by reaction with a carbodiimide (e.g., 1,3-dicyclohexylcarbodiimide, DCC), hydroxysuccinimide, carbonyldiimidazole, or a combination thereof.

Any carboxyl-containing polymer may be used in the method of the invention. In one embodiment, the carboxyl-containing polymer is a condensation polyester.

In a preferred embodiment, the condensation polyester comprises units having the formula:

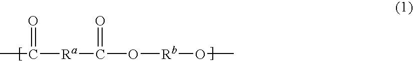

In formula (1), R^{a} and R^{b} independently represent a hydrocarbon group having a minimum of one carbon atom. Preferably, the hydrocarbon groups contain a maximum of twenty-four carbon atoms. More preferably, the hydrocarbon group contains a maximum of ten, more preferably eight, and more preferably six carbon atoms.

In one embodiment, the hydrocarbon group of R^{a} and/or R^{b} is saturated. The saturated hydrocarbon group can be straight-chained, e.g., a straight-chained alkylene group. Some examples of suitable straight-chained alkylene groups include methylene (—CH₂—), dimethylene (—CH₂CH₂—), trimethylene (—CH₂CH₂CH₂—), tetramethylene, pentamethylene, and hexamethylene groups.

The hydrocarbon group of R^{a} and/or R^{b} can alternatively be saturated and branched, i.e., a branched alkylene group. Some examples of suitable branched alkylene groups include 1-methyl-dimethylene (—CH(CH₃)CH₂—), 1-methyl-trimethylene (—CH(CH₃)CH₂CH₂—), 1,1-dimethyl-trimethylene (—C(CH₃)₂CH₂CH₂—), 1,2-dimethyl-trimethylene, 2,2-dimethyl-trimethylene, 1-methyl-tetramethylene, 2-methyl-tetramethylene, 3-methyl-pentamethylene, and the like.

The hydrocarbon group of R^{a} and R^{b} can alternatively be saturated and cyclic, i.e., a cycloalkylene group. The cycloalkylene group may contain three to seven ring carbon atoms. More preferably, the cycloalkylene group contains six ring carbon atoms. Some examples of suitable cycloalkylene groups include 1,4-cyclohexylene and 2,3,5,6-tetramethyl-1,4-cyclohexylene.

The hydrocarbon group of R^{a} and R^{b} can also be unsaturated. The unsaturated hydrocarbon group can be, for example, a straight-chained or branched alkenylene group. Some examples of suitable alkenylene groups include vinylene (—CH═CH—), 1,4-diyl-2-butenylene (—CH—CH═CH—CH—), and 1,4-diyl-2,3-dimethyl-2-butenylene.

The hydrocarbon group of R^{a} and R^{b} can alternatively be unsaturated and cyclic, i.e., a cycloalkenylene group. The cycloalkenylene group preferably contains three to seven ring carbon atoms. Some examples of suitable cycloalkenylene groups include cyclohex-2-en-1,4-diyl, cyclohex-4-en-1,2-diyl, and cyclohexa-2,5-dien-1,4-diyl. The unsaturated cyclic hydrocarbon group can, in addition, be aromatic, i.e., an arylene group. A preferred arylene group is phenylene (e.g., 1,2-phenylene or 1,4-phenylene).

In a preferred embodiment, R^{b} in formula (1) represents a dimethylene group and R^{a} represents a tetramethylene group. In another preferred embodiment, R^{b} in formula (1) represents a dimethylene group and R^{a} represents a phenylene group (as found in commercially available Dacron™ or Mylar™).

The condensation polyester may be prepared by the condensation of one or more diol or polyol compounds, such as ethylene glycol, propylene glycol, or glycerol, with one or more dicarboxyl or polycarboxyl compounds, such as dimethylterephthalate, dimethylphthalate, dimethylmaleate, dimethylfumarate, dimethylglutarate, dimethylmalonate, their acids, and their anhydrides. Accordingly, the condensation polyester contains carboxyl (—C(O)O—) linkages.

In another embodiment, the carboxyl-containing polymer is an addition polymer having pendant carboxyl groups. The addition polymer is preferably derived from one or a combination of acrylate monomers. An acrylate monomer includes at least one carbon-carbon double bond and at least one carboxyl group. A double-double bond and one or more carboxyl groups can be adjacent (as in methyl methacrylate), or alternatively, separated by one or more carbon atoms (as in 3-butenoic acid).

In a preferred embodiment, the acrylate monomers are independently represented by the formula:

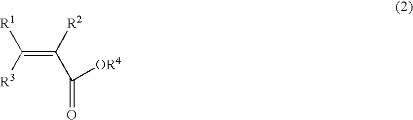

In formula (2), R¹, R², and R³ independently represent hydrogen (H), nitrile (—CN), fluoro (—F), chloro (—Cl), or a hydrocarbon group. R⁴ represents H or a hydrocarbon group.

The hydrocarbon group of R¹, R², R³, and R⁴ has a minimum of one carbon atom. Preferably, the hydrocarbon group contains a maximum of twenty-four carbon atoms. More preferably, the hydrocarbon group contains a maximum of ten, more preferably eight, and even more preferably six carbon atoms.

In one embodiment, the hydrocarbon group of R¹, R², R³, and/or R⁴ is saturated. The saturated hydrocarbon group can be straight-chained, e.g., a straight-chained alkyl group. Some examples of suitable straight-chained alkyl groups include methyl, ethyl, n-propyl, n-butyl, n-pentyl, and n-hexyl groups.

The hydrocarbon group of R¹, R², R³, and/or R⁴ can alternatively be saturated and branched, i.e., a branched alkyl group. Some examples of suitable branched alkyl groups include iso-propyl, iso-butyl, sec-butyl, t-butyl, 1-methylbutyl, 2-methylbutyl, 3-methylbutyl (isopentyl), 1,1-dimethylpropyl, 1,2-dimethylpropyl, 2,2-dimethylpropyl (neopentyl), 1-methylpentyl, 2-methylpentyl, 3-methylpentyl, and 4-methylpentyl groups.

The hydrocarbon group of R¹, R², R³, and/or R⁴ can alternatively be saturated and cyclic, i.e., a cycloalkyl group. The cycloalkyl group preferably contains three to seven ring carbon atoms. Some examples of suitable cycloalkyl groups include cyclopropyl, cyclobutyl, cyclopentyl, cyclohexyl, and cycloheptyl groups.

The hydrocarbon group of R¹, R², R³, and/or R⁴ can also be unsaturated. The unsaturated hydrocarbon group can be, for example, straight-chained, e.g., a straight-chained alkenyl group. Some examples of suitable straight-chained alkenyl groups include vinyl, 2-propen-1-yl, 2-buten-1-yl, 3-buten-1-yl, 2-penten-1-yl, 3-penten-1-yl, 4-penten-1-yl, 2-hexen-1-yl, 3-hexenyl, 4-hexen-1-yl, and 5-hexen-1-yl groups.

The hydrocarbon group of R¹, R², R³, and/or R⁴ can alternatively be unsaturated and branched, e.g., a branched alkenyl group. Some examples of suitable branched alkenyl groups include propen-2-yl, 1-buten-2-yl, 2-buten-2-yl, 1-buten-3-yl, 1-penten-2-yl, 1-penten-3-yl, 1-penten-4-yl, 2-penten-2-yl, 2-penten-3-yl, 2-penten-4-yl, 1-buten-3-methyl-2-yl, 1-buten-3-methyl-3-yl, 2-buten-2-methyl-1-yl, 2-buten-2-methyl-3-yl, 2-buten-2-methyl-4-yl, 2-buten-2-methylenyl, 2-buten-2,3-dimethyl-1-yl, 1-hexen-2-yl, 1-hexen-3-yl, 1-hexen-4-yl, 1-hexen-5-yl, 2-hexen-2-yl, 2-hexen-3-yl, 2-hexen-4-yl, 2-hexen-5-yl, 3-hexen-2-yl, 3-hexen-3-yl, 1-penten-3-methyl-2-yl, 1-penten-3-methyl-3-yl, 1-penten-3-methyl-4-yl, 2-penten-3-methyl-2-yl, and 2-penten-3-methyl-4-yl groups.

The hydrocarbon group of R¹, R², R³, and/or R⁴ can alternatively be unsaturated and cyclic, i.e., a cycloalkenyl group. The cycloalkenyl group preferably contains three to seven ring carbon atoms. Some examples of suitable cycloalkenyl groups include cyclobutenyl, cyclobutadienyl, cyclopentenyl, cyclohexenyl, cyclohexadienyl, cycloheptenyl, and cycloheptadienyl groups.

The unsaturated cyclic hydrocarbon group of R¹, R², R³, and/or R⁴ can, in addition, be aromatic, i.e., an aryl group. Preferably, the aryl group contains six to eighteen ring carbon atoms.

The aryl group can be fused or unfused. A preferred unfused aryl group is phenyl. Some examples of suitable fused aryl groups include naphthyl, phenanthryl, anthracenyl, triphenylenyl, chrysenyl, and pyrenyl.

The hydrocarbon groups described above can include one or more heteroatoms, e.g., nitrogen, oxygen, or sulfur atoms. Hydrocarbon chains that have heteroatoms include, for example, —(CH₂CH₂Y²)ₘₗ—, wherein ml represents 1-8, and Y² represents O, S, or NH.

In formula (2), R⁴ can also represent an unshared pair of electrons. When R⁴ represents an unshared pair of electrons, the acrylate monomer contains a negatively charged carboxylate group. The carboxylate group can be counter balanced by any suitable positively charged group, e.g., an ammonium, phosphonium, lithium, or sodium cation.

In a preferred embodiment of the acrylate monomers according to formula (2), R¹ and R³ represent H. Some examples of such acrylate monomers include acrylate (CH₂═CH—COO⁻ or CH₂═CH—COOH), methyl acrylate (CH₂═CH—COOCH₃), ethyl acrylate, n-propyl acrylate, iso-propyl acrylate, n-butyl acrylate, iso-butyl acrylate, sec-butyl acrylate, tert-butyl acrylate, methacrylate (CH₂═C(CH₃)—COO⁻ or CH₂═C(CH₃)—COOH), methyl methacrylate (CH₂═C(CH₃)—COOCH₃), ethyl methacrylate, propyl methacrylate, n-butyl methacrylate, iso-butyl methacrylate, sec-butyl methacrylate, and tert-butyl methacrylate.

Other suitable acrylate monomers include fumaric acid, maleic acid, 3-methacrylic acid, 3,3-dimethylacrylic acid, 2,3-dimethylacrylic acid, 2-fluoroacrylic acid, 3-chloroacrylic acid, 2-cyanoacrylic acid, hydroxylethylacrylate, hydroxylethylmethacrylate, aminoethylacrylate, aminoethylmethacrylate, N,N-dimethylaminoethylmethacrylate, t-butylaminoethylacrylate, and 3-butenoic acid.

The addition polymer can be derived from a single type of acrylate monomer, i.e., a homopolymer. Some examples of suitable homopolymers include poly(acrylic acid), poly(methacrylic acid), poly(methylacrylate), poly(ethylacrylate), poly(n-propylacrylate), poly(iso-propylacrylate), poly(n-butylacrylate), poly(iso-butylacrylate), poly(t-butylacrylate), poly(methylmethacrylate), poly(ethylmethacrylate), poly(n-propylmethacrylate), poly(iso-propylmethacrylate), poly(n-butylmethacrylate), poly(iso-butylmethacrylate), poly(t-butylmethacrylate), and poly(vinyl acetate).

The addition polymer can also be derived from two or more different types of monomers, i.e., a copolymer. At least one of the monomers from which the copolymer is derived is an acrylate monomer.

In one embodiment, the copolymer is derived from a combination of different types of acrylate monomers. Some examples of such copolymers include acrylic acid-methylacrylate, methylacrylate-ethylacrylate, methylacrylate-isopropylacrylate, acrylic acid-methylmethacrylate, methylacrylate-methylmethacrylate, methylacrylate-maleic acid, and methylmethacrylate-fumaric acid copolymers.

In another embodiment, the copolymer is derived from one or a combination of acrylate monomers and one or a combination of non-acrylate vinyl monomers. Some examples of suitable non-acrylate vinyl monomers include ethene (i.e., ethylene, CH₂═CH₂), propylene (CH₂═CH—CH₃), 2-methylpropene (CH₂═C(CH₃)₂), butadiene (CH₂═CH—CH═CH₂), divinylbenzenes, vinyl chloride (CH₂═CHCl), vinylidene chloride (CH₂═CCl₂), vinyl fluoride (CH₂═CHF), vinylidene fluoride (CH₂═CF₂), tetrafluoroethene (CF₂═CF₂), styrene (CH₂═CH—C₆H₅), acrylonitrile (CH₂═CHCN), acrylamide (CH₂═CHC(O)NH₂), isoprene, and chloroprene.

Some examples of copolymers containing non-acrylate units include ethylene-acrylic acid, ethylene-methacrylic acid, ethylene-methylacrylate, ethylene-methylmethacrylate, ethylene-ethylacrylate, ethylene-n-butylacrylate, ethylene-isobutylacrylate, styrene-acrylic acid, acrylamide-methacrylic acid, ethylene-styrene-methacrylic acid, vinyl fluoride-methylmethacrylate, styrene-butadiene-methylacrylate, ethylene-maleic acid, propylene-fumaric acid, ethylene-acrylic acid-methylmethacrylate, ethylene-isobutylacrylate-methacrylic acid, ethylene-n-butylacrylate-acrylic acid, ethylene-vinylacetate, ethylene-vinylacetate-methacrylic acid, ethylene-vinylacetate-monoethyl maleate, and ethylene-methylacrylate-monoethylmaleate.

The copolymers described above can have any distribution of the monomer units. For example, the copolymer can be a random copolymer, an alternating copolymer, a block copolymer, a graft copolymer, or a combination thereof.

The antimicrobial agent is positively charged after displacing a surface hydroxyl leaving group or the hydrogen atom of a surface hydroxyl group. The antimicrobial agent may be positively charged before reacting with the surface hydroxyl group or may assume a positive charge as a result of the reaction.

The antimicrobial agents can be conveniently represented by the formula:

V^{+a}-LZ (3)

In formula (3), V^{+a} is a positively charged group or an uncharged group that becomes positively charged on displacing surface hydroxyl groups or the hydrogen atoms of the surface hydroxyl groups. The superscript +a indicates the total charge of the moiety V. Preferably, the superscript +a represents 0, 1, or 2.

In one embodiment, a is 0 and V^{+a} represents an uncharged tertiary amino group. In another embodiment, V^{+a} represents a singly or doubly charged moiety. As a singly charged moiety, +a in formula 1 represents 1. As a doubly charged moiety, +a represents 2. The singly or doubly charged moiety may, for example, comprise one or two positively charged nitrogen atoms, one or two positively charged phosphorous atoms, or one or two positively charged sulfur atoms.

In a preferred embodiment, V comprises a moiety with two positive charges after being bonded to the surface. The two positive charges may result from two positively charged nitrogen atoms, such as, for example, —⁺NR²-T-NR₂⁺- or 1,4-diazoniabicyclo[2.2.2]octane. Alternatively, the two positive charges may result from two positively charged sulfur atoms, such as, for example, —⁺SR-T-SR⁺- or 1,4-dithioniumcyclohexane. In this embodiment, T represents any of the saturated or unsaturated hydrocarbon chains having 1 to 24 atoms, as described above. Preferably, T represents a saturated alkyl chain having no heteroatoms. The saturated alkyl chain preferably has one to three carbon atoms.

L represents a saturated or unsaturated hydrocarbon chain. The hydrocarbon chains can contain heteroatoms, as described above. Preferably, the chains contain no heteroatoms. More preferably, the chains contain only saturated carbon atoms.

The minimum number of atoms in the chain is 10, preferably 12, and more preferably 14. The maximum number of atoms in the chain is 24, more preferably 18. The optimum number of atoms in the chain is 16.

L may represent hydrocarbon chains that all have the same length. Preferably, all of the hydrocarbon chains L have 12-18 atoms, more preferably 14-16 atoms, even more preferably 16 atoms, most preferably 16 carbon atoms, and optimally 16 saturated carbon atoms.

Alternatively, L may represent a mixture of hydrocarbon chains. Preferably, at least some of the hydrocarbon chains L in the mixture have 12-18 atoms, more preferably 14-16 atoms, even more preferably 16 atoms, most preferably 16 carbon atoms, and optimally 16 saturated carbon atoms.

It is especially desirable for a significant number of hydrocarbon chains L to have 16 atoms. Generally, at least about 10%, preferably at least about 25%, more preferably at least about 50%, most preferably at least about 75%, and optimally at least about 90% of the hydrocarbon chains L in the mixture have 16 atoms, preferably 16 carbon atoms, and more preferably 16 saturated carbon atoms.

Z represents a stable chemical moiety at the end of hydrocarbon chain L. Z can represent, for example, —H, —OH, —SH, —F, —Cl, —Br, —OR, —NQ₂, —HNC(O)Q, or —OC(O)Q, wherein Q independently represents H or any of the saturated or unsaturated hydrocarbon groups having one to twenty-four carbon atoms described above. More preferably, Q represents methyl or ethyl.

In a preferred embodiment, Z represents H. Some examples of LZ groups, when Z represents H, include decyl, undecyl, dodecyl, tridecyl, tetradecyl, pentadecyl, hexadecyl, heptadecyl, octadecyl, nonadecyl, eicosyl, uneicosyl, docosyl, tricosyl, and tetracosyl.

In one embodiment of formula (3), the antimicrobial agent is an uncharged amine represented by the formula:

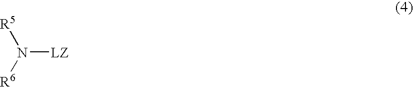

In formula (4), R⁵ and R⁶ independently represent any of the saturated or unsaturated hydrocarbon groups having one to twenty-four carbon atoms, as described above. More preferably, R⁵ and R⁶ independently represent saturated or unsaturated hydrocarbon groups having one to four carbon atoms. Some examples of preferred groups for R⁵ and R⁶ include methyl, ethyl, n-propyl, iso-propyl, n-butyl, iso-butyl, and t-butyl groups. L and Z are as described above for formula (3).

Some examples of antimicrobial agents represented by formula (4) include di-(methyl)decylamine (i.e., (CH₃)₂(C₁₀H₂₁)N), di-(methyl)undecylamine, di-(methyl)dodecylamine, di-(methyl)tridecylamine, di-(methyl)tetradecylamine, di-(methyl)pentadecylamine, di-(methyl)hexadecylamine, di-(methyl)heptadecylamine, di-(methyl)octadecylamine, di-(methyl)nonadecylamine, di-(methyl)eicosylamine, di-(methyl)uneicosylamine, di-(methyl)docosylamine, di-(methyl)tricosylamine, di-(methyl)tetracosylamine, di-(ethyl)hexadecylamine, di-(ethyl)octadecylamine, methylethylhexadecylamine, di-(isopropyl)hexadecylamine, methylisopropylhexadecylamine, di-(methyl)(16-hydroxyhexadecyl)amine, di-(methyl)(16-fluorohexadecylamine), and di-(methyl)(16-aminohexadecylamine).

In another embodiment of formula (3), the antimicrobial agent is a charged amine represented by the formula:

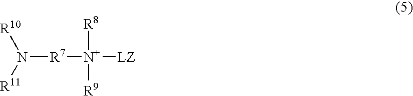

In formula (5), R⁷, R⁸, R⁹, R¹⁰, and R¹¹ independently represent any of the saturated or unsaturated hydrocarbon groups having one to twenty-four carbon atoms, as described above. In addition, R⁸ can optionally join with R¹⁰; and/or R⁹ can optionally join with R¹¹. L and Z have been described above.

Some examples of antimicrobial agents according to formula (5) without linking between R groups include (CH₃)₂N—(CH₂)—N⁺(CH₃)₂(C₁₀H₂₁), (CH₃)₂N—(CH₂)—N⁺(CH₃)₂(C₁₂H₂₅), (CH₃)₂N—(CH₂)—N⁺(CH₃)₂(C₁₆H₃₃), (CH₃)₂N—(CH₂)—N⁺(CH₃)₂(C₁₈H₃₇), (CH₃)₂N—(CH₂)—N⁺(CH₃)₂(C₂₀H₄₁), (CH₃)₂N—(CH₂)—N⁺(CH₃)₂(C₂₂H₄₅), (CH₃)₂N—(CH₂)—N⁺(CH₃)₂(C₂₄H₅₀), (CH₃)₂N—(CH₂CH₂)—N⁺(CH₃)₂(C₁₂H₂₅), (CH₃)₂N—(CH₂CH₂)—N⁺(CH₃)₂(C₁₆H₃₃), (CH₃)₂N—(CH₂CH₂)—N⁺(CH₃)₂(C₁₈H₃₇), (CH₃)₂N—(CH₂CH₂CH₂)—N⁺(CH₃)₂(C₁₆H₃₃), (CH₃)₂N—(CH₂CH₂CH₂CH₂)—N⁺(CH₃)₂(C₁₆H₃₃), (CH₃CH₂)₂N—(CH₂CH₂)—N⁺(CH₃)₂(C₁₆H₃₃), and (CH₃)₂N—(CH₂CH₂)—N⁺(CH₂CH₃)₂(C₁₆H₃₃).

In a further embodiment to formula (5), R⁹ is joined with R¹¹ according to the formula:

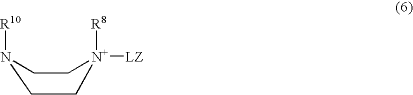

Some examples of antimicrobial agents according to formula (6) include 1,4-dimethyl-4-decyl-piperazin-1-ium, 1,4-dimethyl-4-dodecyl-piperazin-1-ium, 1,4-dimethyl-4-hexadecyl-piperazin-1-ium, 1,4-dimethyl-4-octadecyl-piperazin-1-ium, 1,4-dimethyl-4-eicosyl-piperazin-1-ium, 1,4-dimethyl-4-docosyl-piperazin-1-ium, 1,4-dimethyl-4-tetracosyl-piperazin-1-ium, 1,4-diethyl-4-dodecyl-piperazin-1-ium, 1,4-diethyl-4-hexadecyl-piperazin-1-ium, 1-methyl-4-ethyl-4-hexadecyl-piperazin-1-ium, and 1-propyl-4-methyl-4-hexadecyl-piperazin-1-ium.

In a further embodiment to formula (6), R⁸ is joined with R¹⁰ according to the formula:

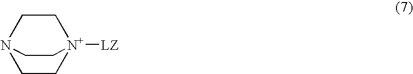

Preferably, L has ten to eighteen carbon atoms, more preferably twelve to sixteen carbon atoms, even more preferably fourteen to sixteen carbon atoms, and most preferably sixteen carbon atoms.

Some examples of antimicrobial agents according to formula (7) include 1-aza-4-azonia-4-decyl-bicyclo[2.2.2]octane, 1-aza-4-azonia-4-undecyl-bicyclo[2.2.2]octane, 1-aza-4-azonia-4-dodecyl-bicyclo[2.2.2]octane, 1-aza-4-azonia-4-tridecyl-bicyclo[2.2.2]octane, 1-aza-4-azonia-4-tetradecyl-bicyclo[2.2.2]octane, 1-aza-4-azonia-4-pentadecyl-bicyclo[2.2.2]octane, 1-aza-4-azonia-4-hexadecyl-bicyclo[2.2.2]octane, 1-aza-4-azonia-4-heptadecyl-bicyclo[2.2.2]octane, 1-aza-4-azonia-4-octadecyl-bicyclo[2.2.2]octane, 1-aza-4-azonia-4-nonadecyl-bicyclo[2.2.2]octane, 1-aza-4-azonia-4-eicosyl-bicyclo[2.2.2]octane, 1-aza-4-azonia-4-uneicosyl-bicyclo[2.2.2]octane, 1-aza-4-azonia-4-docosyl-bicyclo[2.2.2]octane, 1-aza-4-azonia-4-tricosyl-bicyclo[2.2.2]octane, and 1-aza-4-azonia-4-tetracosyl-bicyclo[2.2.2]octane.

For the purpose of clarity, a charged molecule shown in any of the formulas above must be associated with an oppositely charged counter ion. For example, a positively charged amine according to formula (5), (6), or (7) can be ascribed the symbol (I). By necessity, (1) needs to be associated with a negatively charged counteranion or counteranions (W) to make a neutral compound.

Any counteranion may be useful for the purposes of the present invention. A particular counteranion may be chosen for any number of reasons, including its optimizing effect on the method described herein, solubility effects, cost, and so on.

The counteranion may be singly negatively charged, i.e., a simple counteranion. Some examples of suitable simple counteranions include chloride, perchlorate, sulfate, nitrate, and tetrafluoroborate. The counteranion can also be, for example, doubly negatively or triply negatively charged, i.e., a dianion or trianion. Some examples of suitable counter dianions include carbonate, oxalate, fumarate, terephthalate, malonate, and succinate. Some examples of suitable counter trianions include phosphate, citrate, and ascorbate.

From the above, it is evident that numerous ion-counteranion combinations are possible. Accordingly, the ion-counteranion combinations are appropriately described by the following formula:

[I^{+z}]ᵤ[W^{-r}]ₜ (8)

In formula (8), z is the charge of the charged amine and r is the charge of the counteranion. By the rules of chemistry, counter charges are balanced in formula (8) according to the formula uz=rt.

In this invention, carboxyl-containing surfaces are rendered resistant to microbial growth. Some of the microbes which can be resisted include single cell organisms, e.g., bacteria, fungi, algae, and yeast, and mold.

The bacteria can include both gram positive and gram negative bacteria. Some examples of Gram positive bacteria include, for example, Bacillus cereus, Micrococcus luteus, and Staphylococus aureus. Some examples of Gram negative bacteria include, for example, Escherichia coli, Enterobacter aerogenes, Enterobacter cloacae, and Proteus vulgaris. Strains of yeast include, for example, Saccharomyces cerevisiae.

Examples have been set forth below for the purpose of illustration and to describe the best mode of the invention at the present time. However, the scope of this invention is not to be in any way limited by the examples set forth herein.

### EXAMPLES

### Example 1

### Preparation of N-hexadecyl-N,N-dimethyl-N-(2 thiomethyl)ethylammonium bromide

The ammonium salt N-hexadecyl-N,N-dimethyl-N-(2-thiomethyl)ethylammoniumbromide is prepared by adding 66.1 g (0.210 mol) of 1-bromohexadecane in 150 ml of ethyl acetate to 25 g (0.210 mol) of N,N-dimethyl-N-(2-thiomethyl)ethylamine in 250 ml of ethyl acetate. The solution mixture is stirred. The resultant precipitate is collected by suction filtration and washed with ether and dried under vacuum.

### Example 2

### Preparation of 1-hexadecyl-1-thionium-4-thiacyclohexane bromide

The sulfonium salt 1-hexadecyl-1-thionium-4-thiacyclohexane bromide is prepared by adding 63.3 g (0.201 mol) of 1-bromohexadecane in 150 ml of ethyl acetate to 25 g (0.201 mol) of 1,4-dithiane in 250 ml of ethyl acetate. The solution mixture is stirred. The resultant precipitate is collected by suction filtration and washed with ether and dried under vacuum.

### Example 3

### Reductive Pre-Treatment of Polyester Cloth

Strips (18 of size 1″×6″) of white 100% polyester cloth of total weight 2.89 g were placed in a round bottom flask (250 mL) and to them was added absolute ethanol (100 mL) in which sodium borohydride (0.93 g) had been dissolved. With stirring, the reaction mixture was heated at 76° C. for 33 minutes following which the reaction was quenched by the addition of ammonium chloride (1.38 g) dissolved in water (50 mL). The cloth was washed repeatedly with distilled water and air dried.

### Example 4

### Preparation of Antimicrobial Polyester Cloth with 4-hexadecyl-1-aza-4-azoniabicyclo[2.2.2]octane chloride

The resultant strips were treated to render them antimicrobial by a two-step procedure involving treatment with tosyl chloride in aqueous bicarbonate solution, followed, after water washing, with 1-aza-4-azonia-4-hexadecyl-bicyclo[2.2.2]octane chloride in aqueous ethanol. The strips were then washed with water and air dried.

Thus, whereas there have been described what are presently believed to be the preferred embodiments of the present invention, those skilled in the art will realize that other and further embodiments can be made without departing from the spirit of the invention, and it is intended to include all such further modifications and changes as come within the true scope of the claims set forth herein.

We claim:

1. A method for rendering a carboxyl-containing polymer surface resistant to microbial growth, the method comprising:
(i) reacting said carboxyl-containing polymer surface with a metal borohydride in a solvent system comprising a water-soluble alcohol having one to three hydroxy groups and one to four carbon atoms and optionally up to about fifty percent water, in a temperature range having a minimum of about 60° C. and a maximum of about 80° C., and for an amount of time sufficient for reducing carboxyl groups on said carboxyl-containing polymer surface to surface hydroxymethyl groups without altering the nature of the bulk of the polymer; and either
(iia) converting hydroxy groups of said surface hydroxymethyl groups to leaving groups and displacing said leaving groups with one or a combination of antimicrobial agents; or
(iib) displacing the hydrogen atoms of the hydroxy groups of said surface hydroxymethyl groups with one or a combination of antimicrobial agents;
said antimicrobial agents being positively charged after displacing said leaving groups or the hydrogen atoms of said hydroxy groups.

2. The method according to claim 1, wherein said water-soluble alcohol is selected from methyl, ethyl, or isopropyl alcohol.

3. The method according to claim 1, wherein said carboxyl-containing polymer is a carboxyl-containing fabric.

4. The method according to claim 1, wherein said carboxyl-containing polymer is a condensation polyester.

5. The method according to claim 4, wherein said condensation polyester comprises units having the formula 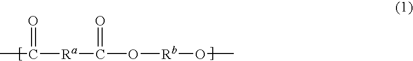
wherein R^{a} and R^{b} independently represent a saturated or unsaturated hydrocarbon group having one to twenty-four carbon atoms.

6. The method according to claim 5, wherein R^{a} and R^{b} independently represent a hydrocarbon group having one to six carbon atoms.

7. The method according to claim 6, wherein R^{b} represents a dimethylene group.

8. The method according to claim 7, wherein R^{a} represents a tetramethylene group.

9. The method according to claim 7, wherein R^{a} represents a phenylene group.

10. The method according to claim 1, wherein said carboxyl-containing polymer is an addition polymer having pendant carboxyl groups.

11. The method according to claim 10, wherein said addition polymer is derived from one or a combination of acrylate monomers independently represented by the formula: 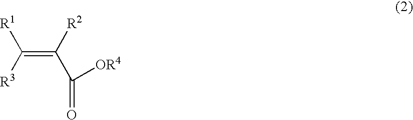
wherein R¹, R², and R³ independently represent hydrogen, a saturated or unsaturated hydrocarbon group having one to twenty-four carbon atoms, nitrile, fluoro, or chloro group; and R⁴ represents H, a saturated or unsaturated hydrocarbon group having one to twenty-four carbon atoms, or an unshared pair of electrons.

12. The method according to claim 11, wherein R¹, R², and R³ independently represent H, or a saturated or unsaturated hydrocarbon group having one to six carbon atoms; and R⁴ represents H, a saturated or unsaturated hydrocarbon group having one to six carbon atoms, or an unshared pair of electrons.

13. The method according to claim 12, wherein R¹ and R³ represent H.

14. The method according to claim 13, wherein said acrylate monomers are selected from acrylate, methyl acrylate, ethyl acrylate, n-propyl acrylate, iso-propyl acrylate, n-butyl acrylate, iso-butyl acrylate, sec-butyl acrylate, tert-butyl acrylate, methacrylate, methyl methacrylate, ethyl methacrylate, propyl methacrylate, n-butyl methacrylate, iso-butyl methacrylate, sec-butyl methacrylate, and tert-butyl methacrylate.

15. The method according to claim 14, wherein said acrylate monomers comprise methyl methacrylate.

16. The method according to claim 11, wherein said addition polymer is a homopolymer.

17. The method according to claim 11, wherein said addition polymer is a copolymer.

18. The method according to claim 17, wherein said copolymer is derived from one or a combination of acrylate monomers and one or a combination of non-acrylate vinyl monomers.

19. The method according to claim 18, wherein said non-acrylate vinyl monomer is ethene.

20. The method according to claim 17, wherein said copolymer is a random copolymer.

21. The method according to claim 17, wherein said copolymer is an alternating copolymer.

22. The method according to claim 17, wherein said copolymer is a block copolymer.

23. The method according to claim 17, wherein said copolymer is a graft copolymer.

24. The method according to claim 1, wherein said metal borohydride is an alkali metal borohydride.

25. The method according to claim 24, wherein said alkali metal borohydride is sodium borohydride.

26. The method according to claim 1, wherein said solvent system comprises ethanol and water.

27. The method according to claim 1, wherein said sufficient amount of time is in a range of about fifteen minutes to thirty-five minutes for a temperature of about 80° C. and in a range of about two hours to four hours for a temperature of about 60° C.

28. The method according to claim 1, wherein said antimicrobial agent is represented by the formula
V^{+a}-LZ (3)
wherein V is a positively charged group or a group that becomes positively charged on displacing surface hydroxyl groups or the hydrogen atoms of said surface hydroxyl groups; a represents 0, 1 or 2; L represents a saturated or unsaturated hydrocarbon chain having ten to twenty-four carbon atoms; and Z represents —H, —OH, —SH, —F, —Cl, —Br, —OR, —NQ₂, —HNC(O)Q, or —OC(O)Q, wherein Q independently represents H or a saturated or unsaturated hydrocarbon group having one to twenty-four carbon atoms.

29. The method according to claim 28, wherein a is 0 and V represents an uncharged tertiary amino group.

30. The method according to claim 29, wherein the antimicrobial agent is represented by the formula: 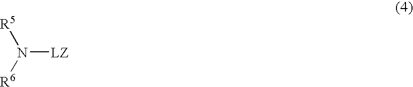
wherein R⁵ and R⁶ independently represent saturated or unsaturated hydrocarbon groups having one to twenty-four carbon atoms.

31. The method according to claim 30, wherein R⁵ and R⁶ independently represent saturated or unsaturated hydrocarbon groups having one to four carbon atoms.

32. The method according to claim 28, wherein V contains a charged amino group.

33. The method according to claim 32, wherein said antimicrobial agent is represented by the formula: 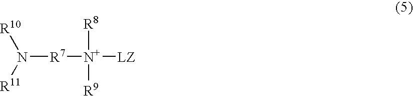
wherein R⁷, R⁸, R⁹, R¹⁰, and R¹¹ independently represent saturated or unsaturated hydrocarbon groups having one to twenty-four carbon atoms; wherein R⁸ can optionally join with R¹⁰; and/or R⁹ can optionally join with R¹¹.

34. The method according to claim 33, wherein said antimicrobial agent is represented by the formula:

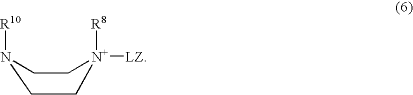

35. The method according to claim 33, wherein said antimicrobial agent is represented by the formula:

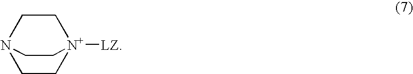

36. The method according to claim 35, wherein L represents a saturated hydrocarbon chain having ten to twenty-four carbon atoms, and Z represents —H.

37. The method according to claim 36, wherein L has ten to eighteen carbon atoms.

38. The method according to claim 37, wherein L has twelve to sixteen carbon atoms.

39. The method according to claim 38, wherein L has fourteen to sixteen carbon atoms.

40. The method according to claim 39, wherein L has sixteen carbon atoms.

41. The method according to claim 1, wherein said surface hydroxyl groups are converted to leaving groups and said leaving groups are displaced under suitable conditions with one or a combination of antimicrobial agents.

42. The method according to claim 41, wherein said surface hydroxyl groups are converted to leaving groups by reacting said surface hydroxyl groups with an activating compound capable of converting said surface hydroxyl groups to ester groups.

43. The method according to claim 42, wherein said activating compound is a sulfonyl chloride.

44. The method according to claim 43, wherein the sulfonyl chloride is benzenesulfonyl chloride, methylsulfonyl chloride, or p-toluenesulfonyl chloride.
